(19) United States
(12) Patent Application Publication
(10) Pub. No.: US 2011/0009616 A1
(43) Pub. Date: Jan. 13, 2011

(54) AGENT FOR PREVENTING ARTERIOSCLEROSIS

(76) Inventors: Akinobu Kishi, Nara (JP); Koji Yuasa, Nara (JP); Taiji Matsukawa, Nara (JP); Takeki Matsui, Nara (JP); Yasumasa Yamada, Nara (JP); Ichiro Yamada, Nara (JP)

Correspondence Address: KRATZ, QUINTOS & HANSON, LLP, 1420 K Street, N.W., 4th Floor, WASHINGTON, DC, 20005, US

(21) Appl. No.: 12/306,364

(22) PCT Filed: Jun. 29, 2007
(86) PCT No.: PCT/JP2007/063094

§ 371 Date: Sep. 22, 2010

(30) Foreign Application Priority Data
Jun. 29, 2006 (JP) 2006-179542

(51) Int. Cl. C07D 487/22

(52) U.S. Cl. 540/145

(57) ABSTRACT

The invention provides antiarterioscierosis agents containing as the active ingredient porphyrin derivatives or ester derivatives thereof as represented by the general formula (1) or (2); and foods or pharmaceutical compositions containing the agents. The porphyrin derivatives and the ester derivatives thereof exhibit an effect of inhibiting the bonding of oxidized LDL to LOX-1 expressed in vascular endothelial cells, macrophage an so on and an antagonistic effect of inhibiting the incorporation of oxidized LDL into cells through LOX-1, and are highly safe. [Chemical formula 1] (wherein R1 and R2 are each a hydrocarbon group having 1 to 4 carbon atoms or the like; and R3 to R8 are each a hydrocarbon group having 1 to 2 carbon atoms, formyl, or the like) [Chemical formula 2] (wherein R1 and R2 are each a hydrocarbon group having 1 to 4 carbon atoms or the like; and R3 to R8 are each a hydrocarbon group having 1 to 2 carbon atoms, formyl, or the like)

### TECHNICAL FIELD

The present invention relates to an agent for preventing arteriosclerosis, an antagonistic agent, and a food or pharmaceutical composition containing the agent.

### BACKGROUND ART

Recently, it has been recognized as the consensus that arteriosclerosis is a symptom positioned a downstream of metabolic syndrome in which adipositas mainly caused by a high-fat diet or insufficient exercise is the uppermost stream, and the progress of the arteriosclerosis forms blood clots to cause heart disease and cerebrovascular disease.

Meanwhile, according to a recent Vital Statistics of the Ministry of Health, Labour and Welfare in Japan, in the number of deaths due to diseases in Japan, malignant neoplasm (tumor and cancer) is the largest number of deaths, heart disease is the second, and cerebrovascular disease is the third, but the sum of deaths due to heart disease and cerebrovascular disease is approximately 28% in the total, and is almost equal to the rate of deaths due to malignant neoplasm of approximately 30% (2005). As for the coping strategy for malignant neoplasm, considering the pathogenic process, the early detection and treatment are important and it is said that a clinical approach is desired. Whereas, as described above, heart disease and cerebrovascular disease positioned the downstream of adipositas and arteriosclerosis caused by lifestyle-related diseases can be approached preventively from foods, and the coping strategy by using daily ingestible forms is desirable.

In arteriosclerosis, the importance of dietetics approaches including dietary habits is as described above, however, in its complicated and various processes, it is important to define which action-mechanism is targeted. Arteriosclerosis is originally caused by the denaturation of low-density lipoprotein (LDL) by the oxidant stress and the like and the dysfunction of vascular endothelium by denatured LDL uptake to vascular endothelial cells, and then as the following sequential process, foam cell formation by differentiation and dedifferentiation of macrophages and vascular smooth muscles is observed. Then finally, through stenotic lesion, blood clots are formed. Therefore, from the viewpoint of prevention, it is important to suppress the primary lesion such as the lipid denaturation by the oxidant stress and the like or the dysfunction by the denatured LDL uptake to the vascular endothelial cells.

In apolipoprotein E-deficient mice, it is reported that, if SR-A (Class A scavenger receptor), a scavenger receptor of macrophage, is knocked out, arteriosclerosis is suppressed but a blood cholesterol level increases (for example, Non-patent Document 1). Conversely, it is reported that, if SR-A is overexpressed, the uptake of cholesterol into macrophages is accelerated but arteriosclerosis is not accelerated (for example, Non-patent Document 2). That is to say, the increase of blood cholesterol level and the lipid accumulation to macrophages and the like are the additional events with respect to the development of arteriosclerosis. Furthermore, the serum lipid of half of the cardiovascular disease patients are normal, and it is said that nothing can prevent the denaturation of lipid sufficiently other than vitamin E (for example, Non-patent Document 3). Thus, the improvement of blood lipid cannot prevent and improve all arteriosclerosis.

On the other hand, it is said that, together with the denaturation of lipid, the dysfunction of vascular endothelial cells is regarded as the initial cause of arteriosclerosis, and it is reported that the dysfunction depends on uptake of denatured LDL, especially oxidized LDL, into vascular endothelial cells through LOX-1 (lectin-like oxidized low-density lipoprotein receptor-1), an oxidized LDL receptor (for example, Non-patent Document 4). In human scavenger receptors, at least 8 of types A to H are found (for example, Non-patent Document 5), it is believed that, in the vascular endothelial cells, at least 6-type scavenger receptors including LOX-1 work (for example, Non-patent Document 6). The dysfunction of vascular endothelial cells due to the denatured LDL uptake through these receptors causes increased expressions of various adhesion factors and suppression of nitric oxide production and the like, and leads accumulation and foam cell formation of monocytes and macrophages, or migration, proliferation, and transformation of smooth muscle cells. It is considered that arteriosclerosis is caused by the result.

Since LOX-1 is expressed not only in the vascular endothelial cells but also in the macrophages and the smooth muscle cells concerned with arteriosclerosis as described above (Non-patent Document 7), it attracts much attention as the target factor of arteriosclerosis. For example, in LOX-1 overexpressed apolipoprotein E-deficient mice, it is ascertained that arteriosclerosis develops through the dysfunction of vascular endothelial cells such as the increases of the number of accumulated macrophages, of the expression of adhesion factors, and of atheromatous part, induced by the overexpression of LOX-1 (for example, Non-patent Document 8). As described above, it is supposed that LOX-1 has a great effect on the onset of arteriosclerosis, however, no commercially available pharmaceutical drug is known to inhibit the binding between LOX-1 and denatured LDL.

Meanwhile, in endothelial and smooth muscle cells, so-called caveola, a membrane domain for endocytosis, exists. As a typical endocytosis pathway other than the caveola, so-called clathrin coated pit also exists. Recently, the relation between the caveola and lipid metabolism has been reported, and furthermore the relation with arteriosclerosis has been studied. For example, it is reported that the endocytosis through caveola is concerned with not only the transports of albumin (for example, Non-patent Document 9) and insulin (for example, Non-patent Document 10) but also the transport of LDL or denatured LDL (for example, Non-patent Document 11). The caveola has caveolin-1 as the coat protein, when a genetically caveolin-1-deficient mouse and an apolipoprotein E-deficient mouse are crossed to obtain a double-knockout mouse, a high anti-arteriosclerosis action with decreased expression of CD36, one of scavenger receptors, is observed (for example, Non-patent Document 12). Therefore, it is supposed that the development of the foods and pharmaceutical drugs targeting the suppression of the lipoprotein transport into cells through caveola leads to the utilization of an agent for preventing arteriosclerosis similar to the LOX-1 antagonist. Accordingly, if a compound can inhibit them simultaneously, the compound can be expected to be an exceptional agent for preventing arteriosclerosis.

Meanwhile, compounds having porphyrin skeleton are widely distributed as metabolites of various living species. As precursors eight molecules of δ-aminolevulinic acids form a cyclic structure to build a tetrapyrrole skeleton, and chlorophyll, a component for photosynthesis, and heme, a component of hemoglobin, are derived from protoporphyrin IX at a downstream. Furthermore, from uroporphyrinogen III, a metabolic intermediate at an upstream, not only the compounds mentioned above but also vitamin B12 and the like are synthesized.

In plants, chlorophyll is also a release source of ROS (reactive oxygen species) and is metabolized aggressively with not only leaf senescence but also tissue damage, and simultaneously, it is also suggested that the metabolic process controls a plurality of defence reactions in plants (for example, Non-patent Document 13), and the degradation of chlorophyll by chlorophyllase and Mg-dechelatase forms pheophorbide a through eliminations of phytol and Mg.

Recently, pheophorbide a has been focused as a marker of ABCG2/BCRP (breast cancer resistance protein), one of ABC (ATP-binding cassette) transporters (for example, Non-patent Document 14). The ABCG2 is also a causal factor of multidrug resistance, and the development of an ABCG2 inhibitor using pheophorbide a as a marker leads to the study for developing anticancer substances. Furthermore, it is said that pheophorbide a is also a causal factor of photosensitivity disease, however, it is a phenomenon caused by the high concentration ingestion (in human, 500 mg/day or higher (Non-patent Document 15), in rat, 220 mg/kg or higher (Non-patent Document 16)), and it is said that current chlorella products with a mixing amount of 60 to 80 mg or less in 100 g has no problem. Actually, in rats, the accumulation of pheophorbide a to skin is low in the case of intravenous injection, and it is said that a main accumulation target is a reticuloendothelial system (Non-patent Document 17).
Non-patent Document 1: Suzuki H. et al. Nature (1997); 386: 292-6.
Non-patent Document 2: Van Eck M. et al. Arterioscler Thromb Vasc Biol (2000); 20: 2600-6.
Non-patent Document 3: Ross R. N Engl J Med (1999); 340: 115-26.
Non-patent Document 4: Sawamura T. et al. Nature (1997); 386: 73-7.
Non-patent Document 5: Murphy J. E. et al. Atherosclerosis (2005); 182: 1-15.
Non-patent Document 6: Adachi H. and Tsujimoto M. Prog Lipid Res (2006); 45: 379-404.
Non-patent Document 7: Chen M. et al. Pharmacol Ther (2002); 95: 89-100.
Non-patent Document 8: Inoue K. et al. Circ Res (2005); 97: 176-84.
Non-patent Document 9: Ghitescu L. et al. J Cell Sci. (1986); 102: 1304-11.
Non-patent Document 10: Bendayan M. et al. J Cell Sci. (1996); 109: 1857-64.
Non-patent Document 11: Kim M-J. et al. Atherosclerosis (1994); 108: 5-17.
Non-patent Document 12: Philippe G.F. et al. Arterioscler Thromb Vasc Biol (2003); 24: 98-105.
Non-patent Document 13: Kariola T. et al. Plant Cell (2005); 17: 282-94.
Non-patent Document 14: Robey R.W. et al. Cancer Res (2004); 64: 1242-6.
Non-patent Document 15: Kimura S. et al. Photomed. Photobiol. (1981); 3: 73.
Non-patent Document 16: Matsuura E. et al. Kitasato Arch. Exp. Med. (1988); 61: 201-13.
Non-patent Document 17: Aprahamian M. et al. Anti-Cancer Drug Design (1993); 8: 101-14.

### DISCLOSURE OF THE INVENTION

### Technical Problems to be Solved

In view of the above mentioned problems, the present invention provides an agent for preventing arteriosclerosis and an antagonistic agent which inhibit the binding of oxidized LDL to LOX-1 expressed in vascular endothelial cells and smooth muscle cells and the like, has an antagonistic action having an ability to suppress oxidized LDL uptake into cells through LOX-1, and is highly safe, and a food or pharmaceutical composition containing the agent.

Furthermore, the invention provides the agent for preventing arteriosclerosis and the antagonistic agent which have the multiple effects of an ability to suppress oxidized LDL uptake through other scavenger receptors other than LOX-1 and of suppression of endocytosis of lipoprotein into cells through caveola, and the food or pharmaceutical composition containing the agent.

### Means to Solve the Problems

The present inventors made intensive studies of the components which have an antagonistic action against LOX-1 and have an excellent ability to suppress oxidized LDL uptake through scavenger receptors other than LOX-1 by screening. As a result, the inventors found that the compounds with a porphyrin skeleton have a remarkable antagonistic action and the achieved the invention. Furthermore, the inventors also found that the compounds have an action to suppress lipoprotein uptake into cells by endocytosis.

That is, the summaries of the invention are as follows:
[1] an agent for preventing arteriosclerosis with a lectin-like oxidized low density lipoprotein receptor (LOX-1) antagonistic action, including a porphyrin derivative composition represented by Formula (1):

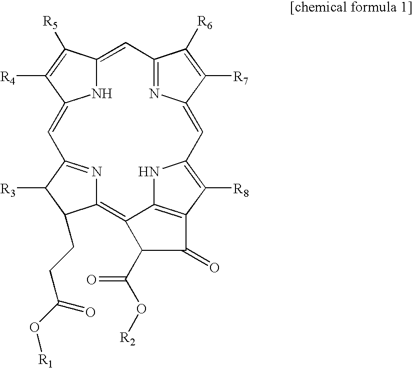

(wherein R₁ and R₂ are each selected from the group consisting of hydrogen and a straight or branched, and saturated or unsaturated hydrocarbon chain having 1 to 4 carbon atoms; and R₃ to R₈ are each selected from the group consisting of hydrogen, a straight, and saturated or unsaturated hydrocarbon group having 1 to 2 carbon atoms, and a formyl group), or an ester thereof;
[2] an agent for preventing arteriosclerosis with a lectin-like oxidized low density lipoprotein receptor (LOX-1) antagonistic action including a porphyrin derivative composition represented by Formula (2):

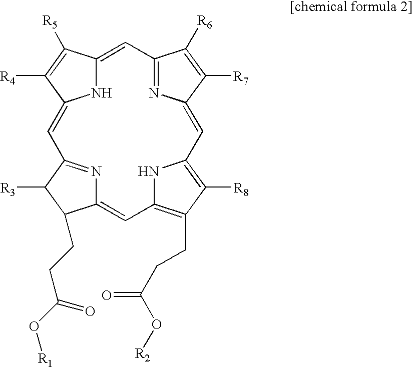

(wherein R₁ and R₂ are each selected from the group consisting of hydrogen and a straight or branched, and saturated or unsaturated hydrocarbon chain having 1 to 4 carbon atoms; and R₃ to R₈ are each selected from the group consisting of a straight, and saturated or unsaturated hydrocarbon group having 1 to 2 carbon atoms, and formyl, carboxymethyl, and carboxyethyl groups), or an ester thereof;
[3] the agent for preventing arteriosclerosis with the lectin-like oxidized low density lipoprotein receptor (LOX-1) antagonistic action according to [1], wherein the porphyrin derivative composition represented by Formula (1) is pheophorbide a or ethyl pheophorbide a;
[4] the agent for preventing arteriosclerosis according to [2], wherein the porphyrin derivative composition represented by Formula (2) is hematoporphyrin or uroporphyrin III;
[5] the agent for preventing arteriosclerosis according to any of [1] to [4], further including an action to suppress oxidized LDL uptake into a cell through a scavenger receptor other than LOX-1;
[6] the agent for preventing arteriosclerosis according to any of [1] to [4], further including an action to suppress oxidized LDL uptake into a cell through caveola;
[7] a lectin-like oxidized low density lipoprotein receptor (LOX-1) antagonistic agent (hereinafter, referred to as LOX-1 antagonistic agent), including a porphyrin derivative represented by Formula (1) or (2), or an ester thereof;
[8] the LOX-1 antagonistic agent according to [7], wherein the porphyrin derivative represented by Formula (1) is pheophorbide a or ethyl pheophorbide a;
[9] the LOX-1 antagonistic agent according to [7], wherein the porphyrin derivative represented by Formula (2) is hematoporphyrin or uroporphyrin III;
[10] the LOX-1 antagonistic agent according to any of [7] to [9], further including an action to suppress oxidized LDL uptake into a cell through a scavenger receptor other than LOX-1;
[11] the LOX-1 antagonistic agent according to any of [7] to [9], further including an action to suppress oxidized LDL uptake into a cell through caveola;
[12] a food composition including the LOX-1 antagonistic agent according to any of [7] to [11];
[13] a pharmaceutical composition including the LOX-1 antagonistic agent according to any of [7] to [11]; and
[14] use of a porphyrin derivative represented by Formula (1) or (2) or an ester thereof for manufacture of a agent for preventing arteriosclerosis.

In the invention, the antagonistic action means, that the binding of oxidized LDL to LOX-1 is inhibited and further the oxidized LDL uptake into cells is inhibited when evaluations are performed by the methods according to Examples described later.

### Effect of the Invention

The compounds of the invention can remarkably inhibit the oxidized LDL uptake into cells through oxidized LDL receptor (LOX-1) deeply concerned with the primary lesion of arteriosclerosis.

### BRIEF DESCRIPTION OF DRAWINGS

FIG. 1 is a graph showing the results of the binding rates of oxidized LDL to bLOX-1 when ethyl pheophorbide a, pheophorbide a, chlorophyll a, chlorophyll b, cyanocobalamin, haemin, hematin, hematoporphyrin, uroporphyrin III, porphobilinogen, pheophytin a, pheophytin b, and Mg2+ are used. The longitudinal axis of the graph shows relative values of the binding amounts of oxidized LDL to LOX-1 fixed on a plate, and the values are obtained by the conversion where the value obtained from the treatment of control without compounds is 100 and the value obtained from the treatment without oxidized LDL is 0. Furthermore, each compound was added so as to be a final concentration of 16 μM.

FIG. 2 is a graph showing the result of a concentration dependence of pheophorbide a having a high antagonistic activity among Examples 1 to 4 described later. DMSO (dimethylsulfoxide; Wako Pure Chemical Industries, Ltd.) with the same concentration is added to each dilution series.

FIG. 3 shows the results of the binding rates of oxidized LDL to hLOX-1. The longitudinal axis of the graph shows relative values of the binding amounts of oxidized LDL to LOX-1 fixed on a plate, and the values are obtained by the conversion where the value obtained from the treatment of control without compounds is 100 and the value obtained from the treatment without oxidized LDL is 0. Furthermore, each compound was added so as to be a final concentration of 16 μM.

FIG. 4 shows the results of the amount of oxidized LDL uptake into the CHO cells expressing bLOX-1 in Example 6. The amount of fluorescence showing the amount of oxidized LDL uptake into the cells is the value obtained by the subtraction of the value of negative control without compounds and fluorescence labeled oxidized LDL in each treatment. Furthermore, the longitudinal axis shows the converted value where the value of the control obtained by the division of the amount of fluorescence by the amount of protein in a solution of cellular lysis is 100%.

FIG. 5 shows the results of the amount of oxidized LDL uptake into the CHO cells expressing hLOX-1 in Example 6. The amount of fluorescence showing the amount of oxidized LDL uptake into the cells is the values obtained by the subtraction of the value of negative control without compounds and fluorescence labeled oxidized LDL in each treatment. Furthermore, the longitudinal axis shows the converted value where the value of the control obtained by the division of the amount of fluorescence by the amount of protein in a solution of cellular lysis is 100%.

FIG. 6 shows the results that each inhibitor was examined on the uptake of each marker of DiI-OxLDL, transferrin as a specific marker of clathrin vesicular transport, and CTB as a transport marker of caveola pathway against each of COS-1 cell, CHO cell expressing bLOX, CHO cell expressing hLOX, COS-1 cell expressing SR-AII, and normal aortic endothelial cell in the same manner as in Example 6 described later. Nystatin was used as a specific inhibitor of CTB.

FIG. 7 shows the results of the ability to suppress oxidized LDL uptake against smooth muscle cells. The fluorescence intensity showing the amount of oxidized LDL uptake into the cells is the value obtained by the division by the number of nuclei dyed with DAPI in the same field of view as the fluorescence intensity from DiI-OxLDL, and shows an average of all 12 fields of view. The control shows the result of the treatment without inhibitor.

### BEST MODE FOR CARRYING OUT THE INVENTION

The porphyrin derivatives composition used in the invention are represented by Formula (1):

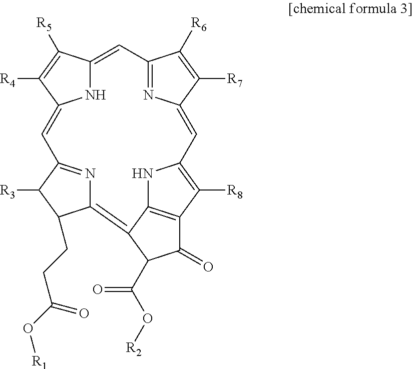

(wherein R₁ and R₂ are each selected from the group consisting of hydrogen and a straight or branched, and saturated or unsaturated hydrocarbon chain having 1 to 4 carbon atoms; and R₃ to R₈ are each selected from the group consisting of hydrogen, a straight, and saturated or unsaturated hydrocarbon group having 1 to 2 carbon atoms, and a formyl group)

or Formula (2):

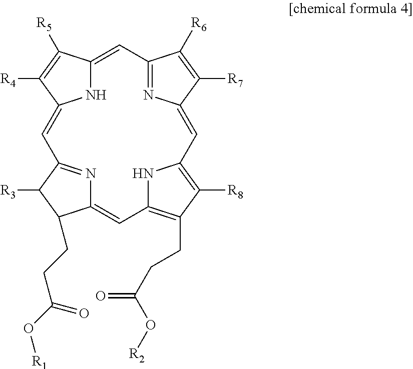

(wherein R₁ and R₂ are each selected from the group consisting of hydrogen and a straight or branched, and saturated or unsaturated hydrocarbon chain having 1 to 4 carbon atoms; and R₃ to R₈ are each selected from the group consisting of a straight, and saturated or unsaturated hydrocarbon group having 1 to 2 carbon atoms, and formyl, carboxymethyl, and carboxyethyl groups).

In Formula (1), from the viewpoint of an excellent effect (especially, the antagonistic action), R₁ is preferably a hydrogen atom or a straight and saturated hydrocarbon group with a carbon number of 1 to 2, and a hydrogen atom or an ethyl group is preferable. R₂, R₃, R₄, R₆, R₇, and R₈ are each preferably a straight and saturated hydrocarbon group with a carbon number of 1 to 2, and a methyl group and an ethyl group are preferable. R₅ is preferably a straight and unsaturated hydrocarbon group with a carbon number of 2 to 4, and an ethylene group is preferable.

In Formula (2), from the viewpoint of an excellent effect (especially, the antagonistic action), R₁ and R₂ are each preferably a hydrogen atom and a straight and saturated hydrocarbon group with a carbon number of 1 to 2, and a hydrogen atom is preferable. Furthermore, R₃, R₄, R₆, and R₈ are each preferably a straight and saturated hydrocarbon group with a carbon number of 1 to 2 and a carboxymethyl group, and a methyl group and a carboxymethyl group are preferable. R₅ and R₇ are each preferably a straight and unsaturated hydrocarbon group with a carbon number of 2 to 4 and a carboxyethyl group, and an ethylene group and a carboxyethyl group are preferable.

For the porphyrin derivatives composition represented by Formula (1), from the viewpoint of an excellent effect (especially, the antagonistic action), pheophorbide a and ethyl pheophorbide a are preferable.

For the porphyrin derivatives composition represented by Formula (2), from the viewpoint of an excellent effect (especially, the antagonistic action), hematoporphyrin and uroporphyrin III are preferable.

The porphyrin derivatives composition represented by Formula (1) or (2) not only have the oxidized LDL receptor (LOX-1) antagonistic action but also inhibit the oxidized LDL uptake through other scavenger receptors, and further have the action to suppress oxidized LDL uptake into cells through caveola, so that the porphyrin derivatives composition have the actions of suppression of endothelium dysfunction or anti-arteriosclerosis.

The porphyrin derivatives composition represented by Formula (1) or (2) can be synthesized from commercially available materials such as chlorophyll by known methods.

In the invention, the porphyrin derivatives composition represented by Formula (1) or (2) include esters. Examples for esters are methyl, ethyl, propyl, and butyl esters.

Furthermore, from the viewpoint that the arteriosclerosis inhibition effect is more improved, the porphyrin derivatives composition represented by Formula (1) or (2) are preferably the compounds which also have the action to suppress the oxidized LDL uptake into cells through scavenger receptors other than LOX-1. Here, the scavenger receptors other than LOX-1 are receptors other than LOX-1, existing in vascular endothelial cells, macrophages, and smooth muscle cells and the like, and receptors bindable with LDL for the uptake into the cells. These scavenger receptors are roughly classified into types A, B, C, D, E, F, G, and H such as SR-AI/II, SR-B1, CD36, MARCO, CD68, SRECs, SR-PSOX, and FEEL-1, but are not limited thereto specifically.

In the invention, as described later in Examples, when the amount of inhibitory activity of oxidized LDL uptake into cells is larger than the amount of inhibitory activity converted from the rate of LOX-1 in the scavenger receptors, the oxidized LDL uptake into cells through the scavenger receptors other than LOX-1 is considered to be inhibited.

In the invention, the porphyrin derivatives composition represented by Formula (1) or (2) and the like are used, but these porphyrin derivatives composition have different antagonist activities from thoseof porphyrin compounds having similar structures such as chlorophyll, cyanocobalamin, haemin, hematin, and porphobilinogen. The reason why the antagonist activities are different in the porphyrin compounds as described above is not clear, but, for example, in the case of the porphyrin derivatives composition represented by Formula (1), since the carbon number of ester chain is shorter than that of other similar compounds, it is supposed that the shorter the ester chain is, the more improved the activity is.

Furthermore, in the case of the porphyrin derivatives composition represented by Formula (2), since a metal atom is not chelated with the porphyrin ring, it is supposed that the chelate of the metal atom is not needed for the activity expression.

Furthermore, as described later in Examples, pheophorbide a also inhibits the oxidized LDL uptake into cells through caveola.

The agent for preventing arteriosclerosis or the LOX-1 antagonistic agent of the invention contains the porphyrin derivatives composition represented by Formula (1) or (2), and may contain only the porphyrin derivatives composition or may further contain other components not inhibiting the antagonist activity. The other components are not limited specifically.

Furthermore, the agent for preventing arteriosclerosis or the LOX-1 antagonistic agent of the invention may be mixed in compositions such as foods and pharmaceutical products by using a known art. In this case, the agent may be used for the composition without treatment or may be mixed in various carriers. Types of the carriers are not limited specifically and may be set appropriately, for example, the carriers for oral administration such as tablet, capsule, candy, gummy candy, and drink are preferred from the viewpoint of easy mixing in foods and the like. However, since it is said that pheophorbide a is also the causal factor of the photosensitivity disease, it is desirable that the mixing amount is 100 mg or less in 100 g of the composition.

Furthermore, for the foods, a desired amount of the compound may be added to various food materials and treated with a general producing method to produce common foods, or the compound without treatment or with treatment for an eater-friendly shape may be used as health foods or functional foods.

Furthermore, examples of the pharmaceutical product are tablet, powder, granule, capsule, suppository, injection, and liquid, and these may be manufactured according to general methods with addition of extender, diluent, lubricant, disintegrator, binder, and flavoring agents and the like.

A daily dose of the agent for preventing arteriosclerosis or the LOX-1 antagonistic agent in these compositions varies according to symptoms, heights, weights, and ages and the like, but it is preferred that the composition is administered to a subject at once or in several times so that the intake per adult would be less than 0.1 mg/kg per day.

The targeting LOX-1 in the invention exists not only in vascular endothelial cells of human beings and non-human mammals (for example, mammals and the like, such as monkey, bovine, swine, equine, sheep, goat, donkey, camel, rabbit, dog, cat, rat, mouse, guinea pig, fowl, duck, and goose) but also in macrophages and smooth muscle cells, and it is supposed that the uptake of oxidized LDL into each cell plays an important role on the onset of arteriosclerosis. Accordingly, since the agent for preventing arteriosclerosis or the LOX-1 antagonistic agent of the invention has the remarkably inhibitory action of the oxidized LDL uptake in a plurality of cell groups such as vascular endothelial cells concerned with arteriosclerosis, the multiple and remarkable suppression of arteriosclerosis is expected, and the agent is highly safe, so that the prevention effect of arteriosclerosis is also expected.

Hereinafter, the present invention will be described in detail with reference to Examples, but is not limited to these Examples.

### Example

The samples used in Examples were prepared as follows.

1. cDNAs of bLOX-1 and hLOX-1

cDNAs of the bLOX-1 and the hLOX-1 were cloned according to the method in the Non-patent Document 4.

Namely, as for bLOX-1, a clone isolated from a cDNA library of vascular endothelial cells was integrated into an expression vector for a temporary expression in COS-7. Then, after co-culture with DiI-OxLDL, DiI positive cells were sorted with a cell sorter to collect plasmids. This screening method was repeated 3 times to isolate a single clone.

Furthermore, as for hLOX-1, amplification was carried out with random primers and oligo dT primers from a cDNA library of human lung. By a positive screening using a full-length bLOX cDNA, hLOX cDNA segments were obtained and then the upstream sequence was obtained by 5′-RACE.

(Insertion to TetOn Vector)

pcDNA31-hLOX1-V5 plasmids (Journal of Molecular and Cellular Cardiology 39 (3) 553-561 (2005)) were digested with PreI, BamHI and subjected to agarose gel electrophoresis, and the gel was cut out and extracted to collect h⁻LOX-1 fragments. pTRE2hyg plasmids (Clontech, TetOn) were digested with EcoRV, BamHI and subjected to agarose gel electrophoresis, and the gel was cut out and extracted to collect Tet response vector fragments. After ligation of the collected h-LOX-1fragments and Tet response vector fragments, the product was transformed into E. coli. The transfected plasmids were collected from E. coli and purified to obtain plasmid DNA as pTRE-hLOX1 (B).

Straight-chained pTRE-hLOX1 (B) by FspI was transfected into CHO-K1 Tet-On cell line (Clontech) by using Lipofectoamine 2000 (manufactured by Invitrogen), and the cell lines were selective-cultivated with hygromycin to form colonies. Single colonies were picked up to obtain 12 cell lines. The expression of each cell line was induced by an addition of doxycycline to the culture medium, each cell extract was prepared, and three cell lines with high induction efficiencies were obtained by western blotting with an anti-V5 antibody.

(Manufacture of SR-AII Expressed Cell Line)

As for cDNA cloning of human SR-AII, the full-length sequence (1,077 base pairs) was obtained by PCR using cDNA derived from human normal lien (Invitrogen) as a template. Additionally a CACC sequence was added to 5′-terminal of the upstream primer. The amplified PCR product was inserted to a pENTR/D-TOPO vector (Invitrogen) and transformed into Mach1 cells (Invitrogen). From the obtained transformant, clones supposed to have the plasmid which was correctly inserted with the SR-AII full-length sequence were selected in usual ways such as a colony PCR method, and the plasmid sequence was sequenced. Furthermore, for the plasmid ascertained to have the correct sequence by sequencing, a site-specific recombination reaction to pDEST26 (Invitrogen) as an expression vector for animal cells was carried out according to a manual. The obtained pDEST26 vector having the full-length sequence of SR-MI was transformed into COS-1 cells (Health Science Research Resources Bank), and cultivated for 48 hours to obtain a transient expressed cell line.

2. Anti-LOX-1 Antibody

An anti-LOX-1 antibody was manufactured according to the method in the above-mentioned Non-patent Document 4.

Namely, the CHO cells expressing bLOX⁻1 or hLOX-1 were treated with 5 mM of EDTA-PBS (room temperature, for 5 minutes), then suspended in a buffer containing protease inhibitors (25 mM of HEPES (pH 7.4)), 10 mM of magnesium chloride, 0.25 M of sucrose, and protease inhibitors (10 U/mL of aprotinine, 2 μg/mL of pepstatin, 50 μg/mL of leupeptin, and 0.35 mg/mL of APMSF), homogenized with a Potter homogenizer, and centrifuged with low-speed (1,500 rpm, 10 minutes, 4° C.). Then, the supernatant was collected and ultracentrifuged (100,000 g, 1 hour, 4° C.), the precipitated membrane fraction was collected and suspended in a phosphate buffer, and the suspension was kept at −20° C. The suspension was used to manufacture an antibody of bovine or human (antigen).

The obtained cell membrane fraction produced immunity in a normal mouse to prepare a mouse monoclonal antibody against bovine or human LOX-1.

The monoclonal antibodies were prepared according to the usual methods described in “Jikkenn Igaku (supplementary volume), Cell Engineering Handbook” (edited by Toshio Kuroki et al., published by Yodosha Co., Ltd, pp 66-74, 1992) and “The Introduction to Monoclonal Antibody Experimental Procedure” (Tamie Ando et al, published by Kodansha, 1991).

The anti-LOX-1 antibody means a specific antibody of LOX-1 as an antigen and works as an antagonist of LOX-1 by an antigen-antibody reaction.

3. PBS Buffer

A PBS buffer was prepared by mixing of 137 mM of sodium chloride, 2.7 mM of potassium chloride, 10 mM of disodium hydrogen phosphate 12-hydrate, and 1.8 mM of potassium dihydrogen phosphate in purified water and following adjustment of its pH to 7.4 with an appropriate amount of hydrochloric acid.

4. Oxidized LDL

Oxidized LDL was prepared in the following manner. Plasma of healthy persons was placed in a centrifuge tube, potassium bromide was added to adjust the specific gravity to 1.019, then the whole was centrifuged with a Beckman L-80 ultracentrifuge (for 20 hours, 58,000 rpm), and the lower layer was collected in another tube. The volume of the collected layer was measured, and potassium bromide was added to adjust the specific gravity to 1.063. Then, the whole was centrifuged with a Beckman L-80 ultracentrifuge (for 20 hours, 58,000 rpm), and the upper layer was collected in another tube. The collected fraction was dialyzed against a phosphate buffer (the external solution was exchanged more than twice) to obtain the purified human LDL.

In order to prepare the oxidized LDL from the obtained purified LDL, a solution was prepared so that the concentrations of the purified LDL and copper sulfate would be 3 mg/mL and 75 μM, respectively, and incubated in a CO₂ incubator for 20 hours. Then, the solution was dialyzed with the solution of 0.15 M sodium chloride containing EDTA (the external solution was exchanged more than twice) to obtain human oxidized LDL.

### Example 1

Ethyl pheophorbide a was derived from pheophorbide a (manufactured by Frontier Scientific Corporation) using a known method. That is, after stirring pheophorbide a (20 mg) with di-tert-butyl dicarbonate (0.32 mmol) and dimethylaminopyridine (4 mg) for 10 minutes, ethanol (0.32 mmol) was added and the whole was further stirred for 1 hour. The product was purified by normal-phase column chromatography with chloroform-methanol (25:1) to obtain ethyl pheophorbide a 6 mg (33%).

### Test Example 1

A LOX-1 antagonist activity of the agent for preventing arteriosclerosis obtained in the above-mentioned Example was evaluated on a LOX-1 binding inhibition rate to oxidized LDL using a recombinant LOX-1 protein obtained from E. coli.

As for the recombinant LOX-1, in cDNA of LOX-1 derived from bovine (bLOX-1) or derived from human (hLOX-1), a sequence to the C-terminal removing a transmembrane region was inserted into a pQE30 vector having a 6-histidine tag sequence (6×His) at the N-terminal (manufactured by Qiagen) so as to have a sequence aligned to a translation frame. The vector was transformed into E. coli able to be controlled by T5 promoter and the expression of each LOX-1 was induced under induction of 1 m M of isopropyl-1-thio-β-D-galactosidase (IPTG). After the induction of each expression at 37° C. for 3 hours, since the recombinant LOX-1 was obtained from an insoluble fraction, E. coli was lysed in a buffer containing 6 M guanidine hydrochloride and purified by affinity column chromatography using a Ni-NTA agarose column (manufactured by Qiagen) as the marker of 6×His (solvent: phosphoric acid buffer). Furthermore, each of the elution fractions containing LOX-1 was dialyzed in a 20 mM tris buffer (pH 9.0) and a 8M urea containing buffer, applied to a HiTrap Q column, an anion exchanger, (manufactured by Amersham), and eluted and purified with a linear concentration gradient of NaCl. Each of the fractions containing LOX-1 obtained above was reduced in a buffer containing excess amount of DTT (dithiothreitol), and then intramolecular S-S bonds were unwinded by a redox system using a mixture of oxidized (GSH) and reduced (GSSG) low molecular thiol compounds. Each LOX-1 obtained above has a binding ability to the oxidized LDL and will be used as a LOX-1 standard for the following plate assays.

The plate assay was carried out using MaxiSorp Immuno Plate (96-well type, manufactured by NUNC). Each LOX-1 purified above was prepared with a PBS buffer so as to be 5 μg/mL, and 100 μL of the solution was applied to each well. After leaving at 4° C. overnight, each well was washed with 400 μL of a PBS buffer twice (400 μL×2) and 300 μL of a PBS buffer containing 25% BlockAce (manufactured by Dainippon Sumitomo Pharma Co., Ltd.) was applied to each well. After leaving at 4° C. overnight, each well was washed with 400 μL of a PBS buffer twice (400 μL×2), a solution of ethyl pheophorbide a was prepared with a PBS buffer containing 1% bovine serum albumin (BSA) so as to have an appropriate dilution rate, and 100 μL of the solution was applied to each well. After leaving at 4° C. overnight, each well was washed with 400 μL of a PBS buffer three times (400 μL×3), a solution of oxidized LDL was prepared with a PBS buffer so as to be 5 μg/mL, and 100 μL of the solution was applied to eachr well. After leaving at 4° C. overnight, each well was washed with 400 μL of a PBS buffer three times (400 μL×3), a horseradish peroxidase-conjugated anti-apolipoprotein B antibody (manufactured by The Binding Site) was diluted 1000 times with a PBS buffer, and 100 μL of the solution was applied to each well. After leaving at room temperature for 2 hours, each well was washed with 400 μL of a PBS buffer five times (400 μL×5), and 100 μL of a 3,3′,5,5′-tetramethylbenzidine (TMB) peroxidase-enzyme immunoassay (EIA)-substrate-kit reagent (manufactured by Bio-Rad) was applied to each well. After an appropriate reaction period, 50 μL of 2M H₂SO₄ was applied to each well to stop the reaction. Finally, a LOX-1 antagonist activity (an oxidized LDL binding inhibition rate to LOX-1) was determined by detection at 450 nm.

### Example 2

Pheophorbide a (manufactured by Frontier Scientific Corporation) was evaluated using a similar method as in Test Example 1.

### Example 3

Hematoporphyrin (manufactured by Wako Pure Chemical Industries, Ltd.) was evaluated using a similar method as in Test Example 1.

### Example 4

Uroporphyrin III (manufactured by Frontier Scientific Corporation) was evaluated using a similar method as in Test Example 1.

### Comparative Example 1

Chlorophyll a (manufactured by Wako Pure Chemical Industries, Ltd.) was evaluated using a similar method as in Test Example 1.

### Comparative Example 2

Chlorophyll b (manufactured by Wako Pure Chemical Industries, Ltd.) was evaluated using a similar method as in Test Example 1.

### Comparative Example 3

Cyanocobalamin (manufactured by Wako Pure Chemical Industries, Ltd.) was evaluated using a similar method as in Test Example 1.

### Comparative Example 4

Haemin (manufactured by Alfa Aesar) was evaluated using a similar method as in Test Example 1.

### Comparative Example 5

Hematin (manufactured by Alfa Aesar) was evaluated using a similar method as in Test Example 1.

### Comparative Example 6

Porphobilinogen (manufactured by Frontier Scientific Corporation) was evaluated using a similar method as in Test Example 1.

### Comparative Example 7

Pheophytin a (manufactured by Wako Pure Chemical Industries, Ltd.) was evaluated using a similar method as in Test Example 1.

### Comparative Example 8

Pheophytin b (manufactured by Wako Pure Chemical Industries, Ltd.) was evaluated using a similar method as in Test Example 1.

### Comparative Example 9

Mg²⁺ (derived from MgCl₂) was evaluated using a similar method as in Test Example 1.

The structure of each compound evaluated in Examples and Comparative Examples is shown below.

The compounds represented by Formula (1) are ethyl pheophorbide a: R₁═R₇═CH₂CH₃, R₂R₃═R₄═R₆═R₈═CH₃, R₅═CH:CH₂; pheophorbide a: R₁═H, R₂═R₃═R₄═R₆═R₈═CH₃, R₅═CH:CH₂, R₇═CH₂CH₃; chlorophyll a: R₁=phytyl, R₂═R₃═R₄═R₆═R₈═CH₃, R₅═CH:CH₂, R₇═CH₂CH₃, N is chelated with Mg; chlorophyll b: R₁=phytyl, R₂═R₃═R₄═R₈═CH₃, R₅═CH:CH₂, R₆═CHO, R₇═CH₂CH₃, N is chelated with Mg; pheophytin a: R₁=phytyl, R₂═R₃═R₄═R₆═R₈═CH₃, R₅═CH:CH₂, R₇═CH₂CH₃; and pheophytin b: R₁=phytyl, R₂═R₃═R₄═R₈═CH₃, R₅═CH:CH₂, R₆═CHO, R₇═CH₂CH₃.

The compounds represented by Formula (2) are haemin: R₁═R₂═H, R₃═R₄═R₆═R₈═CH₃, R₅═R₇═CH:CH₂, N is chelated with Fe and Fe binds to Cl; hematin: R₁═R₂═H, R₃═R₄═R₆═R₈═CH₃, R₅═R₇═CH:CH₂, N is chelated with Fe and Fe binds to OH; hematoporphyrin: R₁═R₂═H, R₃═R₄═R₆═R₈═CH₃, R₅═R₇═CH:CH₂, and uroporphyrin III: R₁═R₂═H, R₃═R₄═R₆═R₈═CH₂COOH, R₅═R₇═CH₂CH₂COOH.

Here, “phytyl” shows CH₂CH═C(CH₃)CH₂CH₂CH₂CH(CH₃)CH₂CH₂CH2CH(CH₃)CH₂CH₂CH₂CH(CH₃)₂.

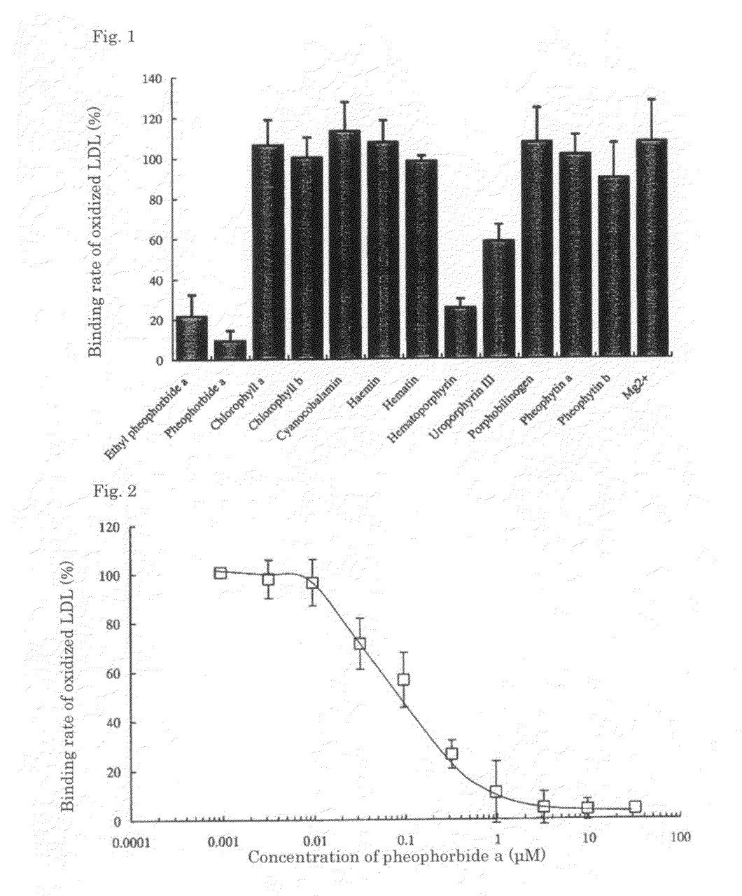

Among the results of Examples 1 to 4 and the Comparative Examples 1 to 9, FIG. 1 shows the results of the binding rates of the oxidized LDL to the bLOX-1. Each inhibitor was evaluated at a final concentration of 16 μM. It is revealed that ethyl pheophorbide a, pheophorbide a, hematoporphyrin, and uroporphyrin III inhibit the binding significantly.

Among them, ethyl pheophorbide a, pheophorbide a, and hematoporphyrin show high inhibition activities.

Furthermore, the concentration dependence of pheophorbide having an extremely high inhibition activity was studied. The result is shown in FIG. 2. From the result, IC50 obtained from the assay was approximately 0.1 μM.

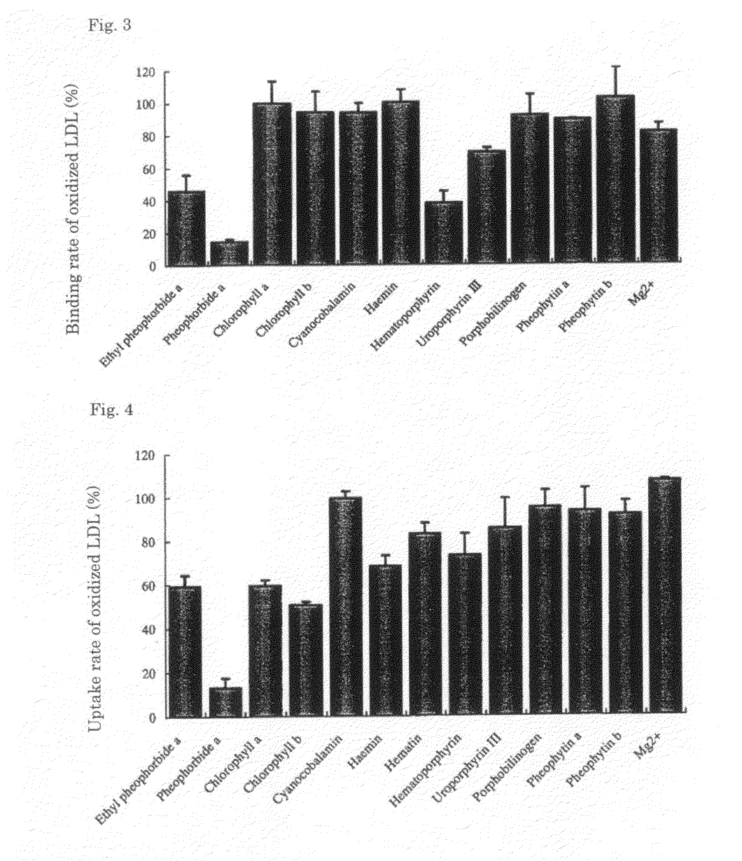

FIG. 3 shows the results of the binding rates of the oxidized LDL to the hLOX-1. Each compound was added so as to be a final concentration of 16 μM. An inhibition manner of the human LOX-1 is similar to that of the bovine LOX-1, and the activities were also ascertained in the LOX derived from human.

### Example 5

In order to examine cytotoxicities of the compounds in Examples 1 to 4 and Comparative Examples 1 to 9, the number of living cells was measured as follows. As a method for measuring the number of living cells, a MTT method using a reduction of MTT (3-(4,5-dimethyl-2-thiazolyl)-2,5-diphenyltetrazolium bromide) to formazan (formed as a poorly soluble precipitate) by dehydrogenase in mitochondria, and the like, are generally used. Thus, “Cell counting Kit-8” (manufactured by Dojindo Laboratories, WST-8 which forms a water soluble formazan is used instead of MTT), a modified method of the MTT method, was used for the measurement of the number of living cells. Specifically, 100 μL of cells were seeded into inner 60 wells of a 96-well plate (manufactured by Falcon) so as to be 3.0×10⁴ cells/mL, respectively (in order to avoid temperature variation, 100 μL of a culture medium was only added into the outer 36 wells). After cultivating for 2 days (37° C.), 50 μL of the culture supernatant was removed and 50 μL of a new culture medium containing each compound with an appropriate dilution rate was added. After adding the new culture medium containing each compound, the cells were cultivated for 7 hours. After 7 hours, each well was washed with 100 μL of the culture medium without the compounds three times, and then 3 μL of a culture medium containing “WST-8”, a “Cell counting Kit-8” solution, was added to each well. After cultivating for 1 hour, absorbance at 450 nm of each culture medium was measured and converted to the number of living cells. It is considered that three washing operations in the process omit the effect of the compounds in the external solution on the absorbance.

### Example 6

As for the same compounds as in Example 5, using Chinese hamster ovary (CHO) cells stably expressing bLOX-1, an ability to suppress fluorescently labeled oxidized LDL uptake into the CHO cells through the bLOX-1 was evaluated. Furthermore, as for the hLOX-1, in the CHO cells using an induction system of tetracycline gene expression, an ability to suppress fluorescently labeled oxidized LDL uptake through the hLOX-1 was evaluated.

As the CHO cells which express the hLOX-1 depending on tetracycline concentrations, the cells transfected with the plasmid described below in a known method were used. The transfected plasmid has a system where a hLOX-1 coding region integrated in pcDNA3.1-hLOX1-V5 (Journal of Molecular and Cellular Cardiology 39 (3) 553-561 (2005)) is mounted in a tetracycline response vector and the expression of hLOX-1 can be controlled depending on the concentration of tetracycline or doxycycline.

An assay using the CHO cells was carried out in the following manner. Cells were seeded into each well of a 24-well plate (manufactured by Falcon) including 500 μL of HamF12 culture medium (manufactured by GIBCO) containing 10% fetal bovine serum (FBS, without tetracycline) so as to be 2.0×10⁴ cells/mL and the cells were cultivated under an environment of 37° C. and 5% CO₂ concentration for 1 day. After cultivating for 1 day, 250 μL of the culture supernatant was removed and 250 μl. of a new culture medium containing doxycycline with a concentration of 200 μg/mL was added (the final concentration of doxycycline was 100 μg/mL). After cultivating for 1 additional day, the culture supernatant was removed and 500 μL of a new culture medium containing each compound with an appropriate dilution rate was added to each well. After adding the new culture medium containing each compound, a pretreatment of 1 hour was carried out under the environment described above to perform a binding reaction of the extract to the LOX-1. Then, the oxidized LDL labeled with 1,1′-dioctadecyl-3,3,3′,3′-tetramethylindocarbocyanine perchlorate (DiI, manufactured by Invitrogen), a carbocyanine fluorescence pigment, was applied to each well so as to be 5 μg/mL, and, under the environment described above, the uptake of the fluorescently labeled oxidized LDL was performed into the CHO cells for 4 hours. After these treatments, each well was washed with 1 mL of a PBS buffer three times and the cells were lysed with 0.1% SDS. After lysis, a certain volume of the lysed solution was collected and dispensed to a 96-well black plate (manufactured by NUNC) to measure the uptaken oxidized LDL using DiI as a marker. “SPECTRA MAX GEMINI EM” of Molecular Devices was used as a spectrofluorometer and the measurement was carried out at an excitation wavelength of 540 nm, a detection wavelength of 585 nm, and a cutoff wavelength of 570 nm.

The CHO cells stably expressing bLOX-1 were cultivated continuously according to the previously reported method (Nature, Vol. 385, p 73-77, 1997). The assay using the cells was carried out in the following manner. The cells were seeded into each well of a 24-well plate (manufactured by Falcon) including 500 μL of HamF12 culture medium containing 10% fetal bovine serum (FBS) so as to be 3.0×10⁴ cells/mL and the cells were cultivated under an environment of 37° C. and 5% CO₂ concentration for two days. After cultivating for two days, 250 μL of the culture supernatant was removed and 250 μL of a new culture medium containing each compound with an appropriate dilution rate was added. After adding the new culture medium containing each compound, a pretreatment of 1 hour was carried out under the environment described above to perform a binding reaction of the extract to the LOX-1. Then, the oxidized LDL labeled with 1,1′-dioctadecyl-3,3,3′,3′-tetramethylindocarbocyanine perchlorate (DiI, manufactured by Invitrogen), a carbocyanine fluorescence pigment, was applied to each well so as to be 5 μg/mL, and under the environment described above the uptake of the fluorescently labeled oxidized LDL was performed into the CHO cells for 6 hours. After these treatments, each well was washed with 1 mL of a PBS buffer three times and the cells were lysed with 0.1% SDS. After lysis, a certain volume of the lysed solution was collected and dispensed to a 96-well black plate (manufactured by NUNC) to measure the uptaken oxidized LDL using DiI as a marker. “SPECTRA MAX GEMINI EM” of Molecular Devices was used as a spectrofluorometer and the measurement was carried out at an excitation wavelength of 540 nm, a detection wavelength of 585 nm, and a cutoff wavelength of 570 nm.

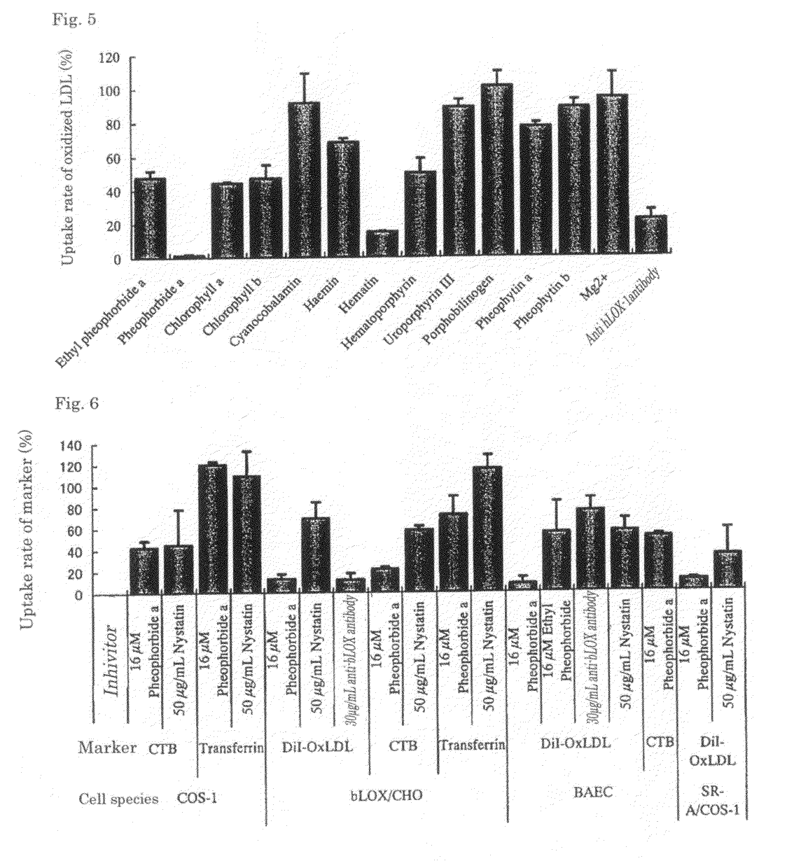

FIGS. 4 and 5 show the results of the amount of oxidized LDL uptake into the CHO cells expressing bLOX-1 and hLOX-1 in Example 6, respectively. Several compounds show activities on both bLOX-1 and hLOX-1 and especially pheophorbide a shows stronger uptake inhibition on hLOX-1 than that of the anti-hLOX antibody.

The results are not shown in Figures, from the cytotoxicity assay using mitochondria activity as an index in Example 5, it is cleared that the inhibition effect is not caused by the cytotoxicity. Similarly, ethyl pheophorbide a, hematoporphyrin, and uroporphyrin III did not show the cytotoxicity in the concentrations where the inhibitory activities were effective.

### Example 7

In each cell of the African green monkey kidney (COS-1) cell (Health Science Research Resources Bank), the CHO cell expressing bLOX, the CHO cell expressing hLOX, the CHO cell expressing SR-AII, and the normal bovine aortic endothelial cell (BAEC, manufactured by Cell Applications Inc.), the inhibition activities of the same compounds as in Examples 1 and 2 on the uptake of the DiI-OxLDL, the uptake of transferrin conjugated with Alexa 568 as a specific marker of a clathrin vesicular transport (an excitation wavelength of 548 nm, a detection wavelength of 605 nm, MolecularProbes), and the uptake of CTB (cholera toxin subunit B) conjugated with Alexa 488 as a transport marker of caveola pathway (an excitation wavelength of 480 nm, a detection wavelength of 530 nm, MolecularProbes) were examined in a similar manner as in Example 6. Nystatin (manufactured by Wako) was used as a specific inhibitor of caveola transport.

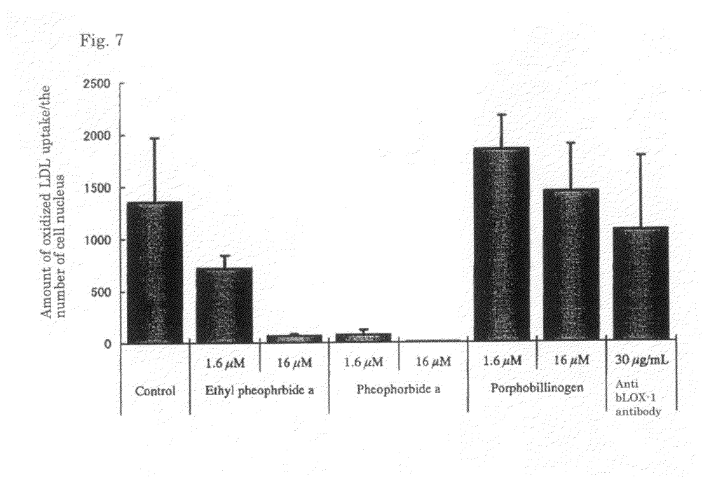

FIG. 6 shows the result of the uptake quantity of each marker obtained from the method of Example 7. From the result of the uptake into the COS-1 cells, since pheophorbide a also inhibits the caveola transport pathway significantly, it can be ascertained that pheophorbide a has the inhibition ability of oxidized LDL uptake into cells through caveola by endocytosis of lipoprotein. Furthermore, since the inhibition of DiI-OxLDL by nystatin in the CHO cells expressing bLOX is nearly equal to that of CTB, it is suggested that the transport pathway of DiI-OxLDL mainly depends on the caveola transport pathway and pheophorbide a has two inhibitory activities of a LOX-1 antagonist activity and a caveola transport inhibition. In the normal bovine aortic endothelial cells, the inhibitory activity of pheophorbide a is stronger than that of the anti-LOX-1 antibody, and the result also suggests that pheophorbide a has the stronger ability to suppress the uptake than the simple antagonistic action. Surprisingly, it is cleared that the pheophorbide a has not only the ability to suppress the oxidized LDL uptake by LOX but also the ability to suppress the oxidized LDL uptake by SR-A. Thus, together with the results of SR-A and endothelium cells, since it is suggested that the pheophorbide a has the ability to suppress the denatured LDL uptake against various scavenger receptors, further activity on the smooth muscle cells which uptake the denatured LDL was studied.

Ethyl pheophorbide a, hematoporphyrin, and uroporphyrin III showed similar inhibition activities to that of pheophorbide a.

### Example 8

As for the compounds in Examples 1 and 2 and Comparative Example 6, the abilities to suppress the oxidized LDL uptake into the smooth muscle cells were evaluated in a similar method to Example 6.

The smooth muscle cells were prepared to 1×10⁵ cells/mL and seeded into a 96-well plate. After cultivating for two days, the cells were treated by each inhibitor in a similar method to Example 6. After the treatment of each inhibitor, the cells were washed with PBS three times and fixed with a 10% formalin solution at room temperature in the dark for 10 minutes. The fixed cells were washed with ultrapure water twice and the nuclei were dyed with DAPI. These cells were analyzed with “IN Cell Analyzer” (manufactured by GE Healthcare Bio-Sciences KK), and the uptake number of Dil-labeled oxidized LDL into the smooth muscle cells was divided by the number of nuclei. Furthermore, 4 fields of view per well were measured and the measurements were n=3 per treatment.

FIG. 7 shows the test results obtained by Example 8. Porphobillinogen was used. As shown in FIG. 7, pheophorbide a shows a very strong ability to suppress the uptake in the smooth muscle cells similar to in the endothelium cells and shows a stronger suppression effect than that of the anti-LOX-1 antibody. Thus, from these results, it is suggested that, similar to in the endothelium cells and the macrophages, in the smooth muscle cells which incorporate the denatured LDL and are deeply concerned with primary lesion of arteriosclerosis, pheophorbide a is a multi-inhibitor because of the abilities of the transport inhibition and of the oxidized LDL uptake suppression through other scavenger receptors along with the LOX-1 antagonist activity.

Hematoporphyrin and uroporphyrin III also showed similar inhibition activities to that of pheophorbide a.

It is preferred that the amount of addition of the agent for preventing arteriosclerosis or the LOX-1 antagonistic agent into food compositions and pharmaceutical compositions is 100 mg or less per 100 g of the composition, and specifically, in consideration of a effective concentration and the like, the addition of 5 to 10 mg of the agent can satisfy both safety and efficacy. For example, mixing examples of the food composition and the pharmaceutical composition are followings.

### Example 9

Mixing Example to Candy

Sugar 64 g and starch syrup 35 g were heated and dissolved at 150° C., and after cooling to 120° C., 1 g of citric acid and 5 to 10 mg of the agent for preventing arteriosclerosis were added and stirred, and then the homogeneous mixture was extruded and cooled to obtain candies.

### Example 10

Mixing Example to Gummy Candy

Sugar 45 g and starch syrup 50 g were heated and dissolved at 110° C., separately swollen and dissolved gelatin 8 g was added, then 2 g of citric acid and 5 to 10 mg of the agent for preventing arteriosclerosis were added and mixed, the mixture was poured into a mold, left for a day, and then unmolded to obtain gummy candies.

### Example 11

Mixing Example to Tablet

Dextrin 20 g, lactose 10 g, palatinose 15 g, potato starch 40 g, magnesium stearate 5 g, and the agent for preventing arteriosclerosis 5 to 10 mg were mixed homogeneously, and the obtained composition was granulated by fluid-bed granulation and dried, and further compressed by a tableting machine to obtain tablets.

### Example 12

Mixing Example to Capsule

Squalene 20 g, linolenic acid triglyceride 20 g, wheat germ oil 10 g, purified sardine oil 20 g, and tocopherol 0.2 g were mixed and stirred to make a uniform composition. Defatted soybean powder 39.8 g and the agent for preventing arteriosclerosis 5 to 10 mg were added to the composition and kneaded by a kneader thoroughly. The obtained kneaded material was capsulated by a capsule filling machine to obtain capsules.

### INDUSTRIAL APPLICABILITY

The agent for preventing arteriosclerosis or the LOX⁻1 antagonistic agent of the present invention has an excellent antagonistic action to inhibit the binding of the oxidized LDL to LOX-1, an oxidized LDL receptor, an excellent action to suppress lipid uptake through LOX-1 and scavenger receptors other than LOX-1, and an excellent ability to inhibit oxidized LDL uptake into cells trough caveola by endocytosis. Furthermore, since the agent inhibits the oxidized LDL uptake to the arteriosclerosis related cells such as vascular endothelial cells continuously, it is expected that the agent suppresses the onset of arteriosclerosis in human beings or non-human animals and relieves the symptoms.

While many advantages of the present invention included in the specification were described above, it will be understood that the disclosure is only an illustrative example in many aspects. Various changes may be made in the details of the invention, especially, as to shape, size, and arrangement without departing from the scope of the invention.

It is to be understood that the scope of the invention is limited to the appended claims.

1. An agent for preventing arteriosclerosis with a lectin-like oxidized low density lipoprotein receptor (LOX-1) antagonistic action, comprising:
a porphyrin derivative composition represented by Formula (1): 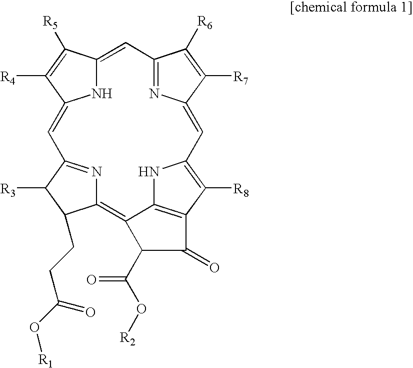
(wherein R₁ and R₂ are each selected from the group consisting of hydrogen and a straight or branched, and saturated or unsaturated hydrocarbon chain having 1 to 4 carbon atoms; and R₃ to R₈ are each selected from the group consisting of hydrogen, a straight, and saturated or unsaturated hydrocarbon group having 1 to 2 carbon atoms, and a formyl group) or an ester thereof.

2. An agent for preventing arteriosclerosis with a lectin-like oxidized low density lipoprotein receptor (LOX-1) antagonistic action, comprising:
a porphyrin derivative composition represented by Formula (2): 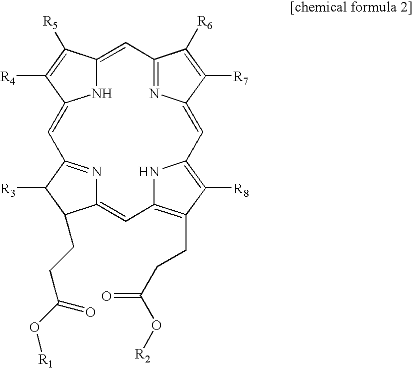
(wherein R₁ and R₂ are each selected from the group consisting of hydrogen and a straight or branched, and saturated or unsaturated hydrocarbon chain having 1 to 4 carbon atoms; and R₃ to R₈ are each selected from the group consisting of a straight, and saturated or unsaturated hydrocarbon group having 1 to 2 carbon atoms, and formyl, carboxymethyl, and carboxyethyl groups) or an ester thereof.

3. The agent for preventing arteriosclerosis according to claim 1, wherein the porphyrin derivative composition represented by Formula (I) is pheophorbide a or ethyl pheophorbide a.

4. The agent for preventing arteriosclerosis according to claim 2, wherein the porphyrin derivative composition represented by Formula (2) is hematoporphyrin or uroporphyrin IR.

5. The agent for preventing arteriosclerosis according to claim 4 further comprising an action to suppress oxidized LDL uptake into a cell through a scavenger receptor other than LOX-1.

6. The agent for preventing arteriosclerosis according to claim 4 further comprising an action to suppress oxidized LDL uptake into a cell through caveola.

7. (canceled)

8. (canceled)

9. A lectin-like oxidized low density lipoprotein receptor (LOX-1) antagonistic action comprising:
a porphyrin derivative composition represented by Formula (1): 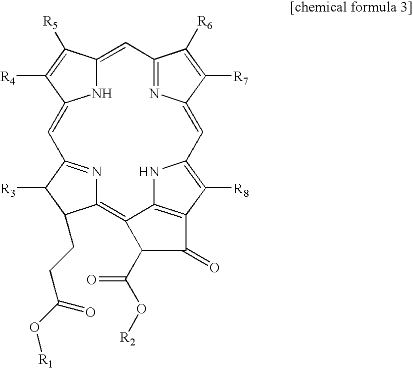
(wherein R₁ and R₂ are each selected from the group consisting of hydrogen and a straight or branched, and saturated or unsaturated hydrocarbon chain having 1 to 4 carbon atoms; and R₃ to R₈ are each selected from the group consisting of hydrogen, a straight, and saturated or unsaturated hydrocarbon group having 1 to 2 carbon atoms, and a formyl group) or Formula (2): 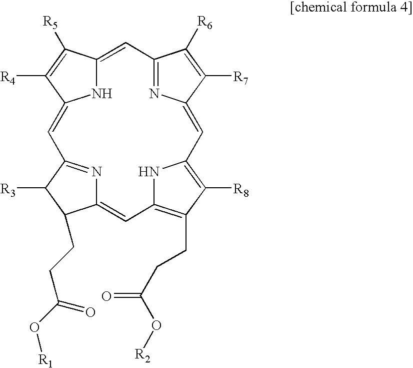
(wherein R₁ and R₂ are each selected from the group consisting of hydrogen and a straight or branched, and saturated or unsaturated hydrocarbon chain having 1 to 4 carbon atoms; and R₃ to R₈ are each selected from the group consisting of a straight, and saturated or unsaturated hydrocarbon group having 1 to 2 carbon atoms, and formyl, carboxymethyl, and carboxyethyl groups) or an ester thereof as an active component.

10. The lectin-like oxidized low density lipoprotein receptor (LOX-1) antagonistic agent according to claim 9, wherein the porphyrin derivative represented by Formula (I) is pheophorbide a or ethyl pheophorbide a.

11. The lectin-like oxidized low density lipoprotein receptor (LOX-1) antagonistic agent according to claim 9, wherein the porphyrin derivative represented by Formula (2) is hematoporphyrin or uroporphyrin III.

12. The lectin-like oxidized low density lipoprotein receptor (LOX-1) antagonistic agent according to claim 11, further comprising an action to suppress oxidized LDL uptake into a cell through a scavenger receptor other than LOX-1.

13. The lectirrlike oxidized low density lipoprotein receptor (LOX-1) antagonistic agent according to claim 11, further comprising an action to suppress oxidized LDL uptake into a cell through caveola.

14. A food composition comprising the lectin-like oxidized low density lipoprotein receptor (LOX-1) antagonistic agent according to claim 13.

15. A pharmaceutical composition comprising the lectin-like oxidized low density lipoprotein receptor (LOX-I) antagonistic agent according to claim 13.

16. Use of a porphyrin derivative represented by Formula (I): 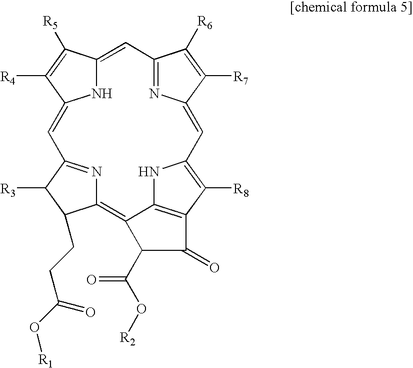
(wherein R₁ and R₂ are each selected from the group consisting of hydrogen and a straight or branched, and saturated or unsaturated hydrocarbon chain having 1 to 4 carbon atoms; and R₃ to R₈ are each selected from the group consisting of hydrogen, a straight, and saturated or unsaturated hydrocarbon group having 1 to 2 carbon atoms, and a formyl group) or Formula (2): 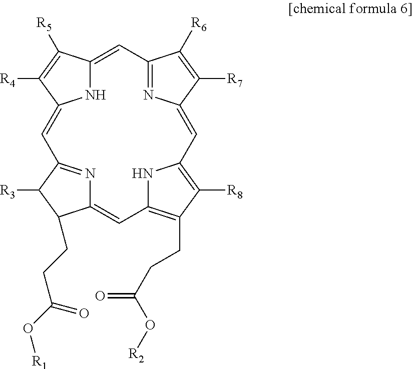
(wherein R₁ and R₂ are each selected from the group consisting of hydrogen and a straight or branched, and saturated or unsaturated hydrocarbon chain having 1 to 4 carbon atoms; and R₃ to R₈ are each selected from the group consisting of a straight, and saturated or unsaturated hydrocarbon group having 1 to 2 carbon atoms, and formyl, carboxymethyl, and carboxyethyl groups), or an ester thereof for manufacture of a agent for preventing arteriosclerosis.

17. The agent for preventing arteriosclerosis according to claim 2, further comprising an action to suppress oxidized LDL uptake into a cell through a scavenger receptor other than LOX-1.

18. The agent for preventing arteriosclerosis according to claim 3, further comprising an action to suppress oxidized LDL uptake into a cell through a scavenger receptor other than LOX-1.

19. The agent for preventing arteriosclerosis according to claim 2, further comprising an action to suppress oxidized LDL uptake into a cell through caveola.

20. The agent for preventing arteriosclerosis according to claim 3, further comprising an action to suppress oxidized LDL uptake into a cell through caveola.

21. The lectin-like oxidized low density lipoprotein receptor (LOX-1) antagonistic agent according to claim 9, further comprising an action to suppress oxidized LDL uptake into a cell through a scavenger receptor other than LOX-1.

22. The lectin-like oxidized low density lipoprotein receptor (LOX-1) antagonistic agent according to claim 10, further comprising an action to suppress oxidized LDL uptake into a cell through a scavenger receptor other than LOX-1.

23. The lectirrlike oxidized low density lipoprotein receptor (LOX-1) antagonistic agent according to claim 9, further comprising an action to suppress oxidized LDL uptake into a cell through caveola.

24. The lectirrlike oxidized low density lipoprotein receptor (LOX-1) antagonistic agent according to claim 10, further comprising an action to suppress oxidized LDL uptake into a cell through caveola.

25. A food composition comprising the lectin-like oxidized low density lipoprotein receptor (LOX-1) antagonistic agent according to claim 9.

26. A food composition comprising the lectin-like oxidized low density lipoprotein receptor (LOX-1) antagonistic agent according to claim 10.

27. A food composition comprising the lectin-like oxidized low density lipoprotein receptor (LOX-1) antagonistic agent according to claim 11.

28. A food composition comprising the lectin-like oxidized low density lipoprotein receptor (LOX-1) antagonistic agent according to claim 12.

29. A pharmaceutical composition comprising the lectin-like oxidized low density lipoprotein receptor (LOX-1) antagonistic agent according to claim 9.

30. A pharmaceutical composition comprising the lectin-like oxidized low density lipoprotein receptor (LOX-1) antagonistic agent according to claim 10.

31. A pharmaceutical composition comprising the lectin-like oxidized low density lipoprotein receptor (LOX-1) antagonistic agent according to claim 11.

32. A pharmaceutical composition comprising the lectin-like oxidized low density lipoprotein receptor (LOX-1) antagonistic agent according to claim 12.
